(19) United States
(12) Patent Application Publication
(10) Pub. No.: US 2006/0003293 A1
(43) Pub. Date: Jan. 5, 2006

(54) Methods for evaluating anticaries efficacy in occlusal surfaces of teeth

(75) Inventor: Robert Vincent Faller, Loveland, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 11/171,786

(22) Filed: Jun. 30, 2005

Related U.S. Application Data
(60) Provisional application No. 60/584,554, filed on Jul. 1, 2004.

(51) Int. Cl. A61K 7/16; A61C 5/00

(52) U.S. Cl. 433/217.1

(57) ABSTRACT

This invention relates to methods for evaluating caries, remineralization, demineralization, and/or erosion in occlusal surfaces of the teeth such as the pits and fissures. Specifically, the present invention relates to an intra oral method of evaluating caries on occlusal surfaces, comprising: a. preparing a dental specimen having at least one closed ended trough; b. preparing a temporary crown or artificial tooth having a well on the occlusal surface; c. placing the dental specimen into the well wherein the trough opening of the dental specimen is facing outward; d. seating the temporary crown or artificial tooth in the oral cavity of the test subject; e. exposing the oral cavity of the test subject to test conditions; f. measuring the degree of caries efficacy on the dental specimen. The methods described herein are especially useful for determining the anticaries efficacy for oral care compositions such as solid, chewable unit dosage forms.

### CROSS REFERENCE TO RELATED APPLICATION

This application claims the benefit of U.S. Provisional Application No. 60/584,554, filed Jul. 1, 2004.

### TECHNICAL FIELD

This invention relates to methods for evaluating caries, remineralization, demineralization, and erosion in occlusal surfaces of the teeth such as the pits and fissures. The methods described herein are especially useful for determining the anticaries efficacy for oral care compositions such as solid, chewable unit dosage forms.

### BACKGROUND ART

Oral care products such as toothpastes are routinely used by consumers as part of their oral care hygiene regimens. These oral care products treat a variety of oral cavity conditions including the treatment and prevention of caries, demineralization, erosion, as well as plaque. These products include, for example, fluoride containing dentifrices used by consumers, concentrated fluoride solutions or gels that are typically applied in the dentist office at periodic, but infrequent, intervals, etc. Despite the availability of these treatments, the occurrence of dental caries and demineralization still remains a problem. While caries rates have been on the decline across a variety of geographies in the past 40 years, worldwide caries remains a prevalent disease that is still the primary cause of tooth loss.

Thus, there remains a need for improved caries prevention or treatment products and treatment regimens. In turn, both in vitro and in vivo methodologies are necessary to test the safety and efficacy of new or improved caries, demineralization, erosion, treatment products.

Furthermore, it is estimated that up to 80-90% of caries occur in the occlusal surfaces. While there are numerous references in the literature to occlusal caries, little has been done with respect to the development of models to mimic occlusal caries and assess the potential benefit of anticaries products against these types of caries. There are a number of reasons for the lack of focus on occlusal caries models. These reasons include the common belief that fluoride dentifrices are not particularly effective against caries on occlusal surfaces. In addition it is difficult to develop an inter oral model that enables specimens to be exposed to normal abrasive and chewing forces. In fact most caries model systems rely on surface demineralization of specimens, resulting in a surface that is weakened relative to sound enamel. Furthermore, there is a belief that relative differences in efficacy for occlusal caries can be demonstrated using standard intraoral model systems, where specimens are protected from abrasive and chewing action via placement in interproximal sites on either an appliance or crown. Other intraoral models for caries evaluation rely on the use of palatal appliances. In these models, subjects are routinely requested to not brush directly on the appliance to avoid potential abrasion, and the device is not subject to chewing forces.

Despite the above known prior art methodologies and technologies for treatment of oral conditions, the prior art has not fully appreciated the benefits of, or solved problems associated with, methods for evaluating anticaries efficacy in occlusal surfaces of the teeth such as the pits and fissures.

### SUMMARY OF THE INVENTION

The present invention relates to an intra oral method of evaluating caries on occlusal surfaces, comprising:
a. preparing a dental specimen having at least one closed ended trough;
b. preparing a temporary crown or artificial tooth having a well on the occlusal surface;
c. placing the dental specimen into the well wherein the trough opening of the dental specimen is facing outward;
d. seating the temporary crown or artificial tooth in the oral cavity of the test subject;
e. exposing the oral cavity of the test subject to test conditions, in one embodiment the test conditions comprise treatment with one or more oral care composition(s);
f. measuring the degree of caries efficacy on the dental specimen.

The present invention further relates to an intra oral method of evaluating caries on occlusal surfaces, comprising:
a. preparing a dental specimen having at least one closed ended trough;
b. inducing demineralization in at least one closed ended trough of the dental specimen;
c. preparing a temporary crown or artificial tooth having a well on the occlusal surface;
d. placing the dental specimen into the well wherein the trough opening of the dental specimen is facing outward;
e. seating the temporary crown or artificial tooth in the oral cavity of the test subject;
f. exposing the oral cavity of the test subject to test conditions, in one embodiment the test conditions comprise treatment with one or more oral care composition(s);
g. measuring the degree of caries efficacy on the dental specimen.

### BRIEF DESCRIPTION OF THE DRAWINGS

The present invention will be better understood by reference to the following detailed description of embodiments in conjunction with the accompanying drawings, in which like reference numerals identify identical elements. Without intending to limit the invention, embodiments of the present invention are described in more detail below.

FIG. 1 is a perspective view of a tooth sample wherein a dental specimen is cut from the incisor tooth sample The dental specimen comprises enamel and dentine.

FIG. 2 is an enlarged partial side elevational view of a cylinder shaped dental specimen having two closed ended troughs.

FIG. 3 is an enlarged partial side elevational view of a cylinder shaped dental specimen having one closed ended trough.

FIG. 4 is an enlarged partial side elevational view of a square dental specimen having two closed ended troughs.

FIG. 5 is an enlarged top planar view of the dental specimen of FIG. 3.

FIG. 6 is an enlarged top planar view of the dental specimen of FIG. 2.

FIG. 7 is an enlarged top planar view of the dental specimen of FIG. 4.

FIG. 8 is an enlarged cross sectional view of the dental specimen of FIG. 2.

FIG. 9 is an enlarged cross sectional view of an artificial molar or a crown for a molar comprising a well. The dental specimen of FIG. 2 is seated in the well.

FIG. 10 is an enlarged top view of the artificial molar or a crown for a molar comprising a well of FIG. 9. The dental specimen of FIG. 2 is seated in the well. The well is in the occlusal cusp of the molar or the crown for a molar.

FIG. 11 is a partial side elevation view of an artificial molar or crown for a molar, having a well and the dental specimen of FIG. 4, wherein the molar or crown is seated between two natural molars along the gum line.

FIG. 12 is a photograph showing a top view of a model of a human subject's full set of lower teeth. One molar has a temporary crown. The crown comprises a well on the occlusal surface of the crown, and a dental specimen having two closed ended troughs is seated in the well. The crown is seated on the tooth surface underneath.

FIG. 13 is a photograph showing a partial denture appliance wherein a dental specimen is seated in a well on the occlusal surface of an artificial molar.

### DETAILED DESCRIPTION

Definitions

As used herein, “comprising” means that other steps and other ingredients which do not affect the end result can be added. This term encompasses the terms “consisting of” and “consisting essentially of”.

By “oral care composition” or “oral composition” as used herein is meant a product which is not intentionally swallowed for purposes of systemic administration of therapeutic agents, but is retained in the oral cavity for a sufficient time to contact some or substantially all of the dental surfaces and/or oral mucosal tissues for purposes of oral activity. In addition these terms can mean a product which may be intentionally swallowed but not swallowed for the purposes of systemic administration of therapeutic agents. Oral care composition includes any product form known in the art including toothpastes, dentifrices, topical oral gels, mouthrinses, denture products, mouthsprays, lozenges, oral tablets, chewable dentifrice tablets, or chewing gums, pet care products, etc. The dentifrice compositions may be a paste, gel, or any configuration or combination thereof. In one embodiment the present invention is particularly suited for evaluation of chewable oral care composition such as those disclosed in Procter & Gamble Copending patent application Ser. Nos. 10/706,103 and 10/706,104, both filed Nov. 12, 2003.

By “caries efficacy” or “caries” as used herein is meant caries, dental erosion, remineralization, and/or demineralization and/or the effect of treatment with an oral care composition, including placebo, on caries, dental erosion, remineralization, and/or demineralization. These oral conditions are further described in WO 02/02096A2, published Jan. 10, 2002, P&G. Dental erosion is further described in Procter & Gamble Copending patent application Ser. No. 10/319,108, filed on Dec. 13, 2002, US Pub. No. 2003/0165442, published Sep. 4, 2003. The present method herein is particularly suited for evaluation of chewable dentifrice oral care composition for anticaries efficacy.

By “test conditions” as used herein is meant the treatment with one or more oral care composition(s) including placebo, exposing the oral cavity to various pH conditions, exposing the oral cavity to foods or beverages, and/or to evaluate the effects of salivary flow, etc.

By “safe and effective amount” as used herein is meant an amount of a component, high enough to significantly (positively) modify the condition to be treated or to effect the desired anticaries result, but low enough to avoid serious side effects (at a reasonable benefit/risk ratio), within the scope of sound medical/dental judgment. The safe and effective amount of a component, will vary with the particular condition (e.g., to effect anticaries activity, to prevent demineralization, or to cause remineralization) being treated, the age and physical condition of the patient being treated, the severity of the condition, the duration of treatment, the nature of concurrent therapy, the specific form employed, and the particular vehicle from which the component is applied.

By “substantially similar” size and shape as used herein is meant that the size and shape of the closed ended troughs varies by no more than about 20%, in another embodiment by no more than about 10%, and in another embodiment by no more than about 5%.

By “test subject” as used herein is meant a human and/or other animal (e.g. pets, zoo, or domestic animal). In one embodiment test subject is human that is in need of a temporary or permanent crown on a molar or premolar. In one embodiment the test subject is a human subject in need of a root canal. In another embodiment the test subject is human or animal subjects who have a missing molar or premolar, for example a partial denture wearer. In these subjects, instead of a crown, an artificial tooth can be inserted into a missing tooth space by attaching the artificial tooth to one or more existing adjacent teeth. The well can be drilled into the artificial tooth into which the dental specimen can be seated.

By “artificial tooth” as used herein is meant a synthetic premolar or molar or a replacement molar or premolar from a human or animal source.

All percentages and ratios used hereinafter are by weight of total composition, unless otherwise indicated.

All measurements made outside the body referred to herein are made at 25° C. unless otherwise specified.

All percentages, ratios, and levels of ingredients referred to herein are based on the actual amount of the ingredient, and do not include solvents, fillers, or other materials with which the ingredient may be combined as a commercially available product, unless otherwise indicated.

All publications, patent applications, and issued patents mentioned herein are hereby incorporated in their entirety by reference. Citation of any reference is not an admission regarding any determination as to its availability as prior art to the claimed invention. To the extent that any meaning or definition of a term in this written document conflicts with any meaning or definition of the term in a document incorporated by reference, the meaning or definition assigned to the term in this written document shall govern.

Preparation of the Dental Specimen

In practicing the present invention a dental specimen must be cut from a tooth sample (e.g. an incisor tooth), then trimmed, and prepared. In one embodiment the dental specimen is cut from the tooth sample, and thereafter trimmed close to the enamel-dentine junction with a dental type saw device, e.g. Leitz Microtome 1600, FRG. The dental specimen may be comprised of enamel, dentine and mixtures thereof, and in one embodiment is comprised only of enamel or only of dentine. The dental specimen may be derived from either a bovine source, a human source, and mixtures thereof, in one embodiment is derived from only human sources. The overall size and shape of the dental specimen may vary, but should be a size (and shape) that is suitable and practical for insertion into the well described herein. The size of the well (and dental specimen) has practical limitations especially when the well is created in a crown of a human test subject. In one embodiment the size of the dental specimen is from about 2 mm to about 4 mm length; from about 2 mm to about 4 mm width, and from about 1 mm to about 5 mm depth. For oval, circular, or elliptical the size of the dental specimen is from about 2 to about 5 mm diameter and from about 1 mm to about 5 mm in depth. In one embodiment the shape is square, rectangular, oval, elliptical, circular, or any other suitable shape.

In order the prepare the dental specimen, the tooth samples are generally analyzed for restorations, caries lesions, white spots, etc. and tooth samples with sound enamel and dentine are selected and cleaned for further processing. Thereafter, dental specimens are cut from the tooth samples. Methods of cutting the tooth samples, tools used for cutting, and subsequent sanding of the dental specimens from the teeth samples are known in the art, see for example, Lagerweij, M. D. Damen, J. J. M. and ten Cate J. M., Demineralization of Dentine Grooves in vitro., Caries Res. 30: 231-236 (1996). For example, Leitz Microtome 1600, FRG is suitable for trimming.

Once the dental specimen is cut (or drilled) and prepared, at least one closed ended trough must be cut into the top surface of the dental specimen. Methods of cutting, tools used for cutting, and subsequent sanding to adjust the size of the trough of the dental specimens are known in the art. In one embodiment the trough is cut with a microdrill, such as a video equipped microdrill manufactured by Stellar Systems, Cincinnati, Ohio. To create the trough, repeated linear drillings are performed with the microdrill to create the closed ended trough of desired length and height. The width of the drill bit will determine the width of the closed ended trough. In one embodiment the width of the drill bit is from about 300 microns to about 500 microns.

The term “closed ended trough” as used herein means that the trough does not extend across the entire top surface of the dental specimen and that the length of the trough does not extend to the outer edges of the dental specimen. The use of closed ended troughs in the present method provides added stability to the dental specimen and minimizes breakage of the dental specimen, minimizes the potential for flow-through effects that are not present in natural tooth surfaces and which are present with open ended troughs. The closed ended troughs also duplicate, as closely as possible, the natural morphology of the tooth surface.

In one embodiment the dental specimen has from about 1 to about 4 closed ended troughs; and in another embodiment the specimen has from about 1 to about 2 closed ended troughs. Again the size and the shape of the trough may vary, but the trough should be a size (and shape) that is suitable and practical for insertion into the well described herein. Since the dental specimen has some practical size limitations, so does the trough(s) described herein. In one embodiment the shape of the closed ended trough is square, rectangular, oval, elliptical, circular, or any other suitable shape. In another embodiment the closed ended trough is rectangular or square.

In one embodiment the size of the closed ended trough is from about 150 microns to about 1,000 microns width and/or length and/or diameter, in another embodiment from about 250 microns to about 800 microns width and/or length and/or diameter; , and from about 200 microns to about 800 microns depth, in another embodiment from about 300 microns to about 500 microns depth.

In one embodiment the closed ended trough has a rectangular shape having from about 200 microns to about 800 microns width, in another embodiment from about 300 microns to about 500 microns width; from about 400 microns to about 1000 microns length, in another embodiment from about 700 microns to about 900 microns length; and from about 200 microns to about 800 microns depth, in another embodiment from about 300 microns to about 500 microns depth.

In one embodiment when the dental specimen has more than one trough, all of the troughs are the same size and shape. In another embodiment the dental specimen comprises trough(s) having different size and shape.

In a dental specimen having more than one trough, the troughs may be arranged in any orientation in relation to each other, such as parallel, perpendicular, etc. In one embodiment all of the troughs in the dental specimen are parallel to one another. In another embodiment the dental specimen has two, parallel, rectangular closed ended troughs having similar dimensions, in another embodiment having substantially similar dimensions. Next the closed ended trough may be cleaned prior to inducing demineralization such as with high pressure air and water for approximately 30 seconds to 1 minute.

Inducing Demineralization in Troughs of the Dental Specimen

When it is desirable to induce demineralization in one or more troughs, a protective coating is applied to either the top surface of the dental specimen or the entire surface of the dental specimen, as described herein below. The protective coating provides a barrier to demineralization or caries formation induced by the demineralization agent(s) and targets the demineralization specifically to the targeted trough(s).

When it is desirable to induce demineralization in one or more troughs, the demineralization is induced by chemical means, biological means, or mixtures thereof (herein referred to as the “demineralization agent”). For example, chemical means for creating artificially induced demineralized lesion in enamel or dentine are disclosed in White et al., Caries Res 21, 228-242, 1987, and include lactic acid solutions, for example a 0.1 M lactic acid solution at a pH of from 4-5 generally applied at 37 degrees C. for a sufficient time generally from 1 to 7 days. In addition biological means for creating artificially induced demineralized lesions in enamel or dentine include inoculating with a bacterial composition such as S. mutans. Other biological means are disclosed in Fontana, et al., An in vitro Microbial Model for Studying Secondary Caries Formation, Caries Res. 30:112-118, 1996.

When it is desirable to prepare a dental specimen having both sound trough(s) and demineralized trough(s), the sound portions of the dental specimen must be protected during exposure to the demineralization agents. In one embodiment, first, one or more troughs are cut into the dental specimen. Thereafter, the protective coating is applied to the entire surface of the dental specimen except for the trough(s). This avoids exposing the rest of the dental specimen surface to the demineralization agents or chemicals, leaving only the trough exposed. Thereafter, the dental specimen is submerged into a solution of the demineralization agent, for example, a solution of lactic acid. Thereafter, the dental specimen is removed from the demineralization agent and rinsed with water. The protective coating may be left or removed. In one embodiment the protective coating is then removed. One or more additional trough(s) can then be drilled or cut into the dental specimen. Since these additional trough(s) are not exposed to the demineralization agent, these trough(s) comprise only sound enamel or dentine.

The protective coating is any material that will adhere to the surface of the dental specimen, be resistant to removal by the demineralization agent and not be reactive with the demineralization agent, and be easily removed after demineralization of the desired trough(s) is accomplished. In one embodiment the protective coating is acid resistant nail polish. In another embodiment the protective coating is a light curing bonding agent such as Scotchbond multipurpose from 3M.

In one embodiment any subsequently added troughs are parallel to the first trough(s). After demineralization the dental specimen is sterilized prior to insertion in the well.

In one embodiment of the present invention, all of the trough(s) in the dental specimen contain only sound (non-demineralized) enamel or dentine. In another embodiment of the present invention, all of the troughs of the dental specimen contain only demineralized enamel or dentine. In yet another embodiment of the present invention, the dental specimen contains at least one sound (non-demineralized) trough and at least one demineralized trough. In one embodiment the dental specimen contains one sound (non-demineralized) trough and one demineralized trough.

In one embodiment the dental specimen contains a sound top surface, at least one sound trough, and at least one demineralized trough. This provides a dental specimen that may withstand the forces of mastication (chewing), withstand the abrasive action of brushing with a toothbrush and withstand the abrasive action from the use of an abrasive containing toothpaste in the oral cavity. Therefore the efficacy of an oral care composition that requires brushing with a toothbrush may be evaluated. This type of dental specimen also allows for the simultaneous evaluation of an oral care composition on both demineralization and remineralization aspects of the caries process.

Preparation of the Well; Placement of the Dental Specimen Into the Well; Seating the Temporary Crown or Artificial Tooth in the Oral Cavity

In practicing the present method it is necessary to prepare a well on the occlusal surface of a temporary crown or artificial tooth of a test subject. The well can be created by any practical means available to the dental practitioner. The overall size and shape of the well may vary, but it should be a size (and shape) that is suitable and practical for insertion into temporary crown or artificial tooth as described herein. The size of the well has practical limitations especially when the well is created in a temporary crown of a human test subject. In one embodiment the size of the well is from about 1 mm to about 5 mm length and/or diameter; from about 1 mm to about 5 mm width and/or diameter; from about 2 mm to about 6 mm deep. In another embodiment the size of the well is from about 2 mm to about 4 mm length and/or diameter; from about 3 mm to about 4 mm width and/or diameter; from about 3 mm to about 4 mm deep. In one embodiment the shape of the well may be square, rectangular, oval, elliptical, circular, or any other suitable shape to accept the dental specimen. In one embodiment the well is cast into a temporary crown by any casting method known to one of skill in the art for casting a temporary or permanent crown, see for example, Shillingburg, Fundamentals of fixed Prosthodontics, 3^{rd} ed. January 1997, and Dunitz, M., Planning and Making Crowns and Bridges, 3^{rd} ed., January 1998.

In another embodiment the well is drilled or cut into an artificial tooth. Methods of cutting, tools used for cutting, and subsequent sanding to adjust the size of the well of the artificial tooth are known in the art. In one embodiment the well is cut with a drill or microdrill of appropriate size drill bits. To create the well, repeated linear drillings may be performed with the drill to create a well of desired length, width, and height. The selection of the width of the drill bit may determine the width of the well in the artificial tooth.

After formation of the well, the dental specimen is placed into the well wherein the top surface of the dental specimen is positioned so that the trough opening is facing outward. In one embodiment the top of the dental specimen is positioned flush with the top of the top outer surface of the temporary crown. Any suitable inert material, for example a wax material or temporary dental cement such as Nogenol® Temporary Cement (manufacturer by G.C. America), may be used to stabilize the dental specimen in the well and to fill any extra volume or space difference between the volume of the dental specimen and the well volume. In one embodiment the inert material is free of fluoride ions.

Lastly, the temporary crown (comprising the well and the dental specimen) is seated onto the molar or premolar, needing the crown, of the test subject. Methods for seating of the temporary crown are well known in the art, for example, Shillingburg, Fundamentals of fixed Prosthodontics, 3^{rd} ed. January 1997, and Dunitz, M., Planning and Making Crowns and Bridges, 3^{rd} ed., January 1998.

If an artificial tooth is used, the dental specimen is placed into the well of the artificial tooth wherein the top surface of the dental specimen is positioned so that the trough opening is facing outward. In one embodiment the top of the dental specimen is positioned flush with the top outer surface of the artificial tooth. Any suitable inert material, for example a wax material or temporary dental cement, may be used to stabilize the dental specimen in the well and to fill any extra volume or space difference between the volume of the dental specimen and the well volume. Thereafter, the artificial tooth (comprising the well and the dental specimen) is seated with an attachment means (e.g. partial denture) along the gum in a missing tooth space. In this instance the test subject may be a person who has a missing molar or premolar, for example a partial denture wearer. In these subjects, instead of a crown, an artificial tooth can be attached with thin wires to one or more existing natural teeth or to the denture, via means known the art. If an artificial tooth is used, the artificial tooth may be seated into a missing tooth opening in the oral cavity along the gumline, see for example, Shillingburg, Fundamentals of fixed Prosthodontics, 3^{rd} ed. January 1997, and Dunitz, M., Planning and Making Crowns and Bridges, 3^{rd} ed., January 1998.

Exposure to Oral Care Composition and Evaluation of Efficacy

In practicing the present method it is necessary to expose the oral cavity of the test subject to one or more oral care composition(s) and thereafter measure the effect relating to caries efficacy, demineralization, remineralization, erosion on the dental specimen. In one embodiment the oral care composition comprises a safe and effective amount of an anticaries agent. In another embodiment the composition is a non-fluoride oral care composition.

In one embodiment the anticaries agent is selected from the group consisting of xylitol, fluoride ion source, and mixtures thereof. The fluoride ion source provides free fluoride ions during the use of the oral care composition. In one embodiment the fluoride ion source is selected from the group consisting of sodium fluoride, stannous fluoride, indium fluoride, organic fluorides such as amine fluorides, and sodium monofluorophosphate. Sodium fluoride is the fluoride ion in another embodiment. Norris et al., U.S. Pat. No. 2,946,725, issued Jul. 26, 1960, and Widder et al., U.S. Pat. No. 3,678,154 issued Jul. 18, 1972, disclose such fluoride salts as well as others that can be used as the fluoride ion source.

In one embodiment the level of fluoride ion source in the oral care composition is from about 5 ppm to about 5,000 ppm, in another embodiment from about 10 ppm to about 3000 ppm, and in another embodiment from about 50 ppm to about 2,800 ppm, and in another embodiment from about 100 ppm to about 2,000 ppm, and in another embodiment from about 300 ppm to about 1,500 ppm, and in even another embodiment from about 850 ppm to about 1,100 ppm.

Exposure of the oral cavity of the test subject to one or more oral care composition(s) may comprise any treatment regimen to be evaluated or known treatment regimen for oral care compositions. For example, a safe and effective amount of the oral care composition may be topically applied to the surface of the teeth, in several conventional ways. For example, the teeth surfaces may be rinsed with a solution (e.g., mouth rinse, mouth spray); or in a dentifrice (e.g., toothpaste, tooth gel or tooth powder), the teeth are bathed in the liquid and/or lather generated by brushing the teeth. Other non-limiting examples include applying a non-abrasive gel or paste, directly to the teeth.

Other treatment regimens include applying a safe and effective amount of an oral care composition to the teeth (for example, by rinsing with a mouth rinse, directly applying a non-abrasive gel with or without a device, chewing a chewable dentifrice tablet, chewing a lozenge, chewing a solid dosage form, applying a dentifrice or a tooth gel with a toothbrush, etc.) for at least about 10 seconds, in another embodiment from about 20 seconds to about 10 minutes, in even another embodiment from about 30 seconds to about 60 seconds. The treatment regimen often involves expectoration of most of the composition following such contact. The frequency of such contact may be from about once per week to about four times per day, in another embodiment from about thrice per week to about three times per day, in even another embodiment from about once per day to about twice per day. The period of such treatment typically ranges from about one day to several years, in another embodiment from about 5 days to about 6 months, in another embodiment from about 14 days to about 60 days.

In one embodiment two treatment regimens are evaluated 1. a toothpaste comprising 1100 ppm of fluoride ion (e.g. sodium fluoride) and 2. a chewable dentifrice that delivers from about 0.2 mg to about 3 mg of fluoride ion during use. Each subject has one temporary crown seated on a molar. The temporary crown has a cylinder shaped well having a 3 mm diameter and a 2 mm depth. Each subject has a cylinder enamel dental specimen (2.5 mm diameter and 1.5 mm depth) having two rectangular troughs (size 500 microns depth and width and 800 microns length) seated in the well. One trough is demineralized and one trough contains only sound enamel. The subjects use the oral care compositions 2-3 times daily for about 1 month. After about 1 month, the temporary crowns are removed from the oral cavity and the dental specimens are removed from the well. The presence of lesions or demineralization in the sound troughs or on the top surface of the dental specimens as well as the degree of caries, erosion, demineralization and/or remineralization of lesions (including the average depth of the lesions) are measured by polarized light microscopy. See for example, Arends et al., Caries Res., In vitro Demineralization of Human Enamel in Artificial U-Shaped Grooves, 20:217-222 (1986); Lagerweij et al., Caries Res., Demineralization of Dentine Grooves in vitro, 30:231-236 (1996); Lagerweij et al., Caries Res., Effect of Fluoridated Toothpaste on Lesion Development in Plaque-Filled Dentine Grooves: An Intra Oral Study, 31:141-147 (1997).

Methods, other than polarized light microscopy, useful for measuring caries, erosion, demineralization and/or remineralization are taught in the art and include transverse microradiography, cross-sectional microhardness, optical fluorescence as well as others.

In another embodiment, after exposure to the treatment regimen, dental specimens are removed from the temporary crown or appliance and analyzed by any relevant technique used to measure changes in enamel or dentin mineral content. In one embodiment, thin cross-sections (80-120 μm thick) are removed from each specimen, with the cross-section taken perpendicular to the orientation of the troughs. Removing cross-sections may be achieved with the Silverstone-Taylor Hard Tissue Microtome (Series 1000 Deluxe), which is capable of producing sections that do not require polishing prior to radiographic analyses. Other techniques enable removal of thicker cross-sections from each dental specimen, which are then polished down to the desired thickness (80-120μ thick) for radiographic analyses. Once the cross sections are prepared, analysis may be conducted with quantitative transverse microradiography (TMR), which enables a quantitative assessment of lesion size, depth and intensity. Polarized light microscopy is also capable of providing an excellent assessment of lesion changes for these type samples. Lesion size, depth, intensity, etc. for each dental specimen may be assessed relative to untreated control dental specimens exposed to the same pre-treatment conditions as test specimens.

The present invention will be better understood by reference to the following description of embodiments in conjunction with the accompanying drawings, in which like reference numerals identify identical elements. Without intending to limit the invention, embodiments of the present invention are described in more detail below. Many variations of these embodiments are possible without departing from the scope of the invention.

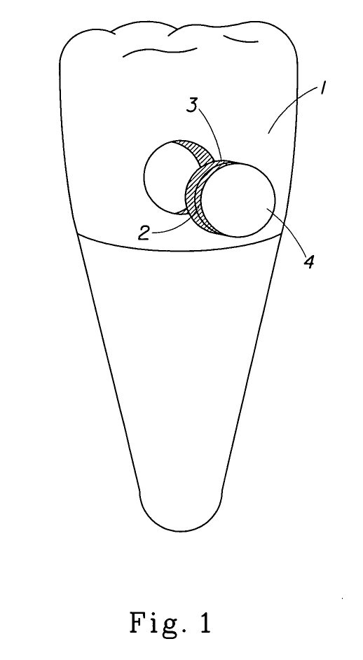

In FIG. 1 a cylinder shaped dental specimen 4 is cut from a human incisor tooth sample 1. The dental specimen 4 is comprised of a dentine layer 3 as well as an enamel layer 2. The dental specimen 4 is cut completely through the incisor tooth sample 1.

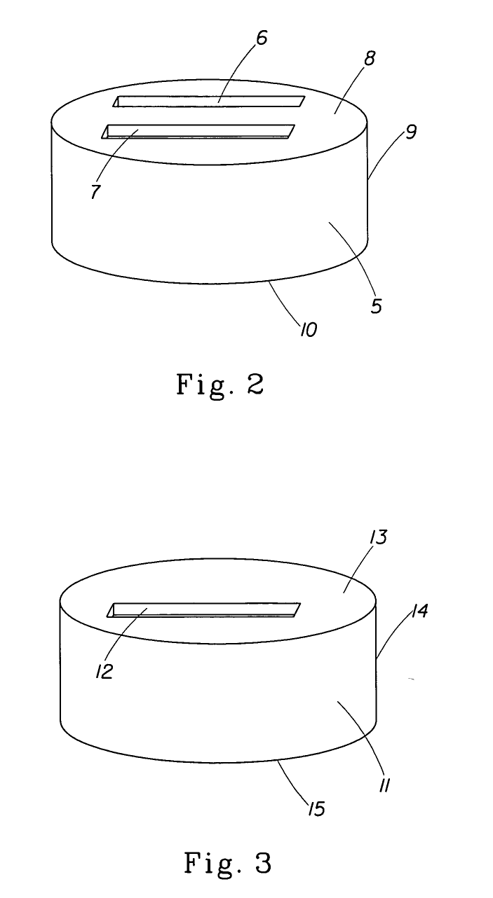

In FIG. 2 a cylinder shaped dental specimen 5 comprises of a rectangular closed ended trough 6 which is parallel to a second closed ended trough 7 which has a substantially similar size and shape as the closed ended trough 6. The dental specimen 5 has a top side 8, a continuous circular side 9, as well as a bottom side 10. The opening of the rectangular closed ended troughs 6 and 7 face outward from the top side 8 of the cylinder shaped dental specimen 5.

In FIG. 3 a cylinder shaped dental specimen 11 comprises a rectangular closed ended trough 12. The cylinder shaped dental specimen 11 has a top side 13, a continuous circular side 14, as well as a bottom side 15. The opening of the rectangular closed ended trough 12 face outward from the top side 13 of the cylinder shaped dental specimen 11.

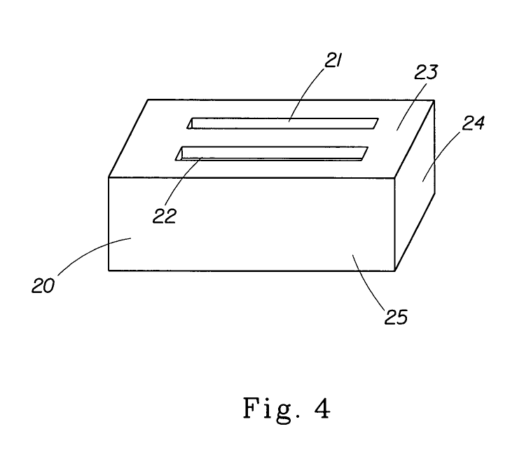

In FIG. 4 a boxed shaped dental specimen 20 comprises of a rectangular closed ended trough 21 which is parallel to a second closed ended trough 22 which has a substantially similar size and shape as the closed ended trough 21. The boxed shaped dental specimen 20 has a top side 23, a side 24, as well as a bottom side 25. The openings of the rectangular closed ended troughs 21 and 22 face outward from the top side 23 of the boxed shaped dental specimen 20.

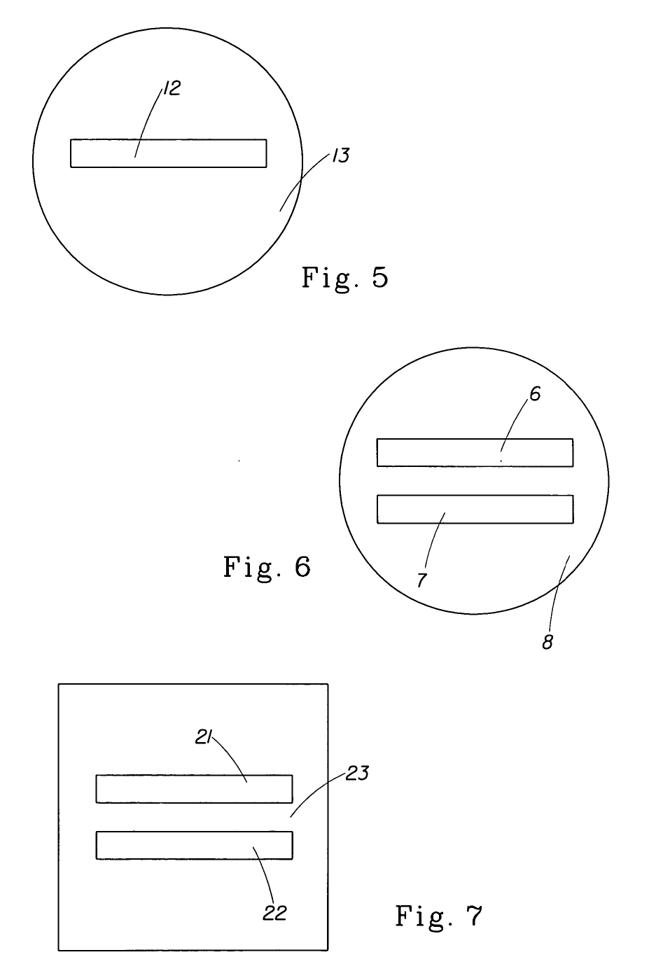

FIG. 5 is a top planar view of the cylinder shaped dental specimen 11 of FIG. 3, comprising a rectangular closed ended trough 12 and a top side 13.

FIG. 6 is a top planar view of the cylinder shaped dental specimen 5 of FIG. 2, comprising two rectangular closed ended troughs 6 and 7, and a top side 8.

FIG. 7 is a top planar view of the boxed shaped dental specimen 20 of FIG. 4, comprising two rectangular closed ended troughs 21 and 22, and a top side 23.

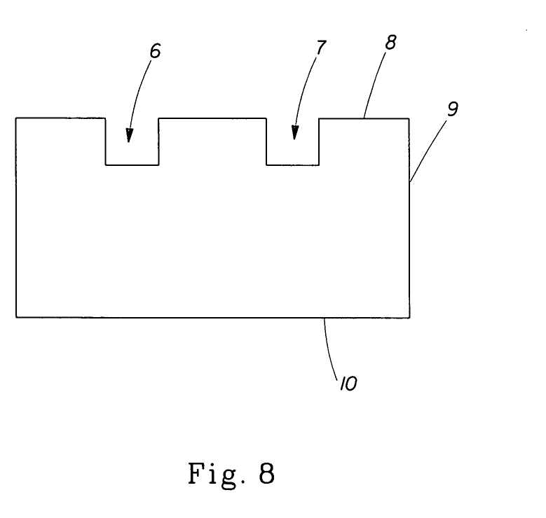

FIG. 8 is a cross sectional view of the cylinder shaped dental specimen 5 of FIG. 2. The cylinder shaped dental specimen 5 comprises two rectangular closed ended troughs 6 and 7, a top side 8, a continuous circular side 9, as well as a bottom side 10. The opening of the rectangular closed ended troughs 6 and 7 face outward from the top side 8 of the cylinder shaped dental specimen 5.

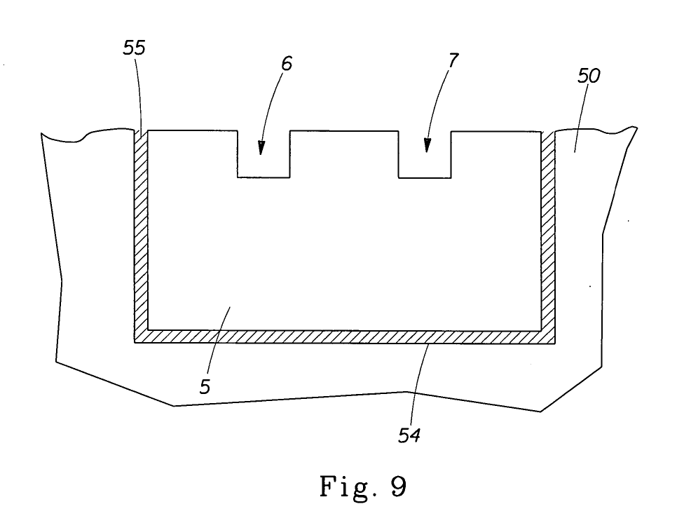

The artificial molar or a crown for a molar 50 of FIG. 9 comprises a well 54. The cylinder shaped dental specimen 5 of FIG. 2 is seated in the well 54. A layer of paraffin wax 55 fills the space between the cylinder shaped dental specimen 5 and the well 54. The cylinder shaped dental specimen has two rectangular closed ended troughs 6 and 7.

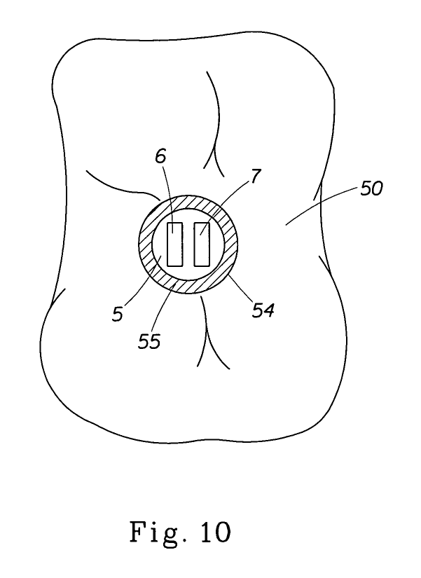

The artificial molar or a crown for a molar 50 of FIG. 10 comprises a well 54. The cylinder shaped dental specimen 5 of FIG. 2 is seated in the well 54. A layer of paraffin wax 55 fills the space between the cylinder shaped dental specimen 5 and the well 54. The cylinder shaped dental specimen has two rectangular closed ended troughs 6 and 7.

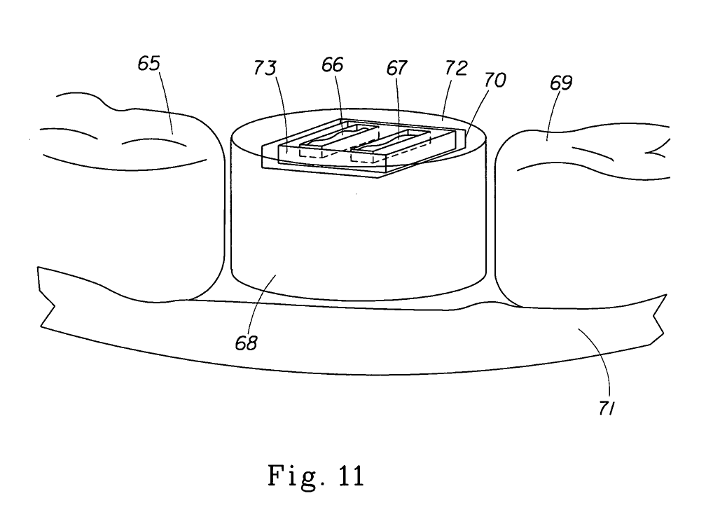

In FIG. 11, the artificial molar or crown for a molar 68, has a well 70 and a rectangular shaped dental specimen 73 seated in the well 70. The rectangular shaped dental specimen 73 has two rectangular closed ended troughs 66 and 67 which have substantially similar size and shape and are parallel to each other. The openings of the rectangular closed ended troughs 66 and 67 face outward from the top side 72 of the rectangular shaped dental specimen 73. The artificial molar or crown 68 is seated between two natural molars, 65 and 69, along the gumline 71.

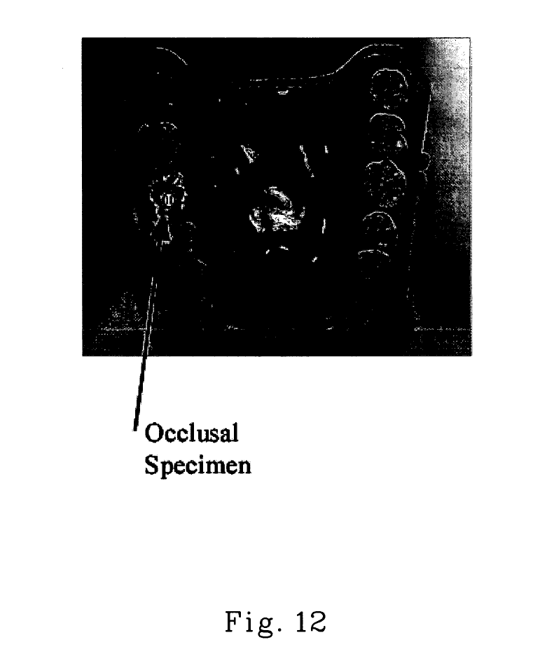

FIG. 12 is a photograph showing a top view of a model of a human subject's full set of lower teeth. One molar has a temporary gold crown. The crown comprises a well on the occlusal surface of the crown, and seated in the well is a dental specimen having two parallel closed ended troughs. The crown is seated on the tooth surface underneath with a removable dental cement.

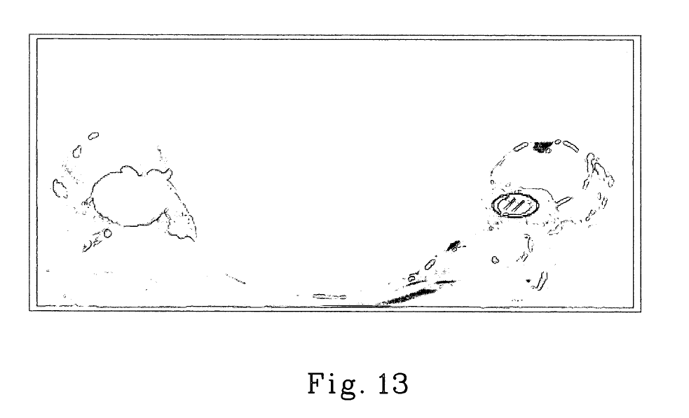

FIG. 13 is a photograph showing a partial denture appliance wherein a dental specimen is seated in a well on the occlusal surface of an artificial molar.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An intra oral method of evaluating caries on occlusal surfaces, comprising:
a. preparing a dental specimen having at least one closed ended trough;
b. inducing demineralization in at least one closed ended trough of the dental specimen;
c. preparing a temporary crown or artificial tooth having a well on the occlusal surface;
d. placing the dental specimen into the well wherein the trough opening of the dental specimen is facing outward;
e. seating the temporary crown or artificial tooth in the oral cavity of the test subject;
f. exposing the oral cavity of the test subject to test conditions;
g. measuring the degree of caries efficacy on the dental specimen.

2. The method of claim 1 wherein the dental specimen has from about 1 to about 4 troughs.

3. The method of claim 2 wherein the dental specimen has 2 troughs.

4. The method of claim 2 wherein the troughs have from about 200 microns to about 800 microns width, from about 400 microns to about 1000 microns length, and from about 200 microns to about 800 microns depth.

5. The method of claim 4 wherein the troughs have from about 300 microns to about 500 microns width, from about 700 microns to about 900 microns length; and from about 300 microns to about 500 microns depth.

6. The method of claim 4 wherein the dental specimen has more than one trough and the size and shape of all of the troughs are substantially similar.

7. The method of claim 2 wherein the dental specimens are comprised of enamel or dentine.

8. The method of claim 7 wherein the dental specimens are derived from human or bovine teeth.

9. The method of claim 2 wherein the dental specimens are derived from human teeth.

10. The method of claim 1 wherein the demineralization is induced in at least one trough of the dental specimen by biological or chemical means.

11. The method of claim 10 wherein the dental specimen has at least one demineralized trough and at least one sound (mineralized) trough.

12. The method of claim 10 wherein the dental specimen has only demineralized trough(s) or only sound (mineralized) trough(s).

13. The method of claim 11 wherein the surface of the dental specimen, after inserted into the oral cavity of the test subject, that is exposed to the oral cavity, is sound.

14. The method of claim 1 wherein the conditions comprise treatment with one or more oral care composition(s).

15. An intra oral method of evaluating caries on occlusal surfaces, comprising:
a. preparing a dental specimen having at least one closed ended trough;
b. preparing a temporary crown or artificial tooth having a well on the occlusal surface;
c. placing the dental specimen into the well wherein the trough opening of the dental specimen is facing outward;
d. seating the temporary crown or artificial tooth in the oral cavity of the test subject;
e. exposing the oral cavity of the test subject to test conditions;
f. measuring the degree of caries efficacy on the dental specimen.
